(19) United States
(12) Patent Application Publication
(10) Pub. No.: US 2013/0211387 A1
(43) Pub. Date: Aug. 15, 2013

(54) APPARATUS FOR OPHTHALMOLOGICAL, IN PARTICULAR REFRACTIVE, LASER SURGERY

(71) Applicants: Peter Riedel, Nurnberg (DE); Christof Donitzky, Eckental (DE)

(72) Inventors: Peter Riedel, Nurnberg (DE); Christof Donitzky, Eckental (DE)

(73) Assignee: WAVELIGHT GMBH, ERLANGEN (DE)

(21) Appl. No.: 13/001,954

(22) PCT Filed: Jun. 30, 2008
(86) PCT No.: PCT/EP08/05334

§ 371 Date: Mar. 22, 2011

(51) Int. Cl. A61F 9/008

(52) U.S. Cl. CPC A61F 9/00804
USPC 606/4

(57) ABSTRACT

In an apparatus for ophthalmological, in particular refractive, laser surgery a pachymetric measuring device based on an optical-coherence interferometric measuring process is controlled in such a way that the position of the measuring beam emitted by the measuring device follows movements of the eye to be treated. The eye movements are registered by means of a camera of an eye-tracker. For the purpose of changing the position of the measuring beam, a partially transmitting deflecting mirror, via which the measuring beam is routed onto the eye, is movably, in particular tiltably, arranged.

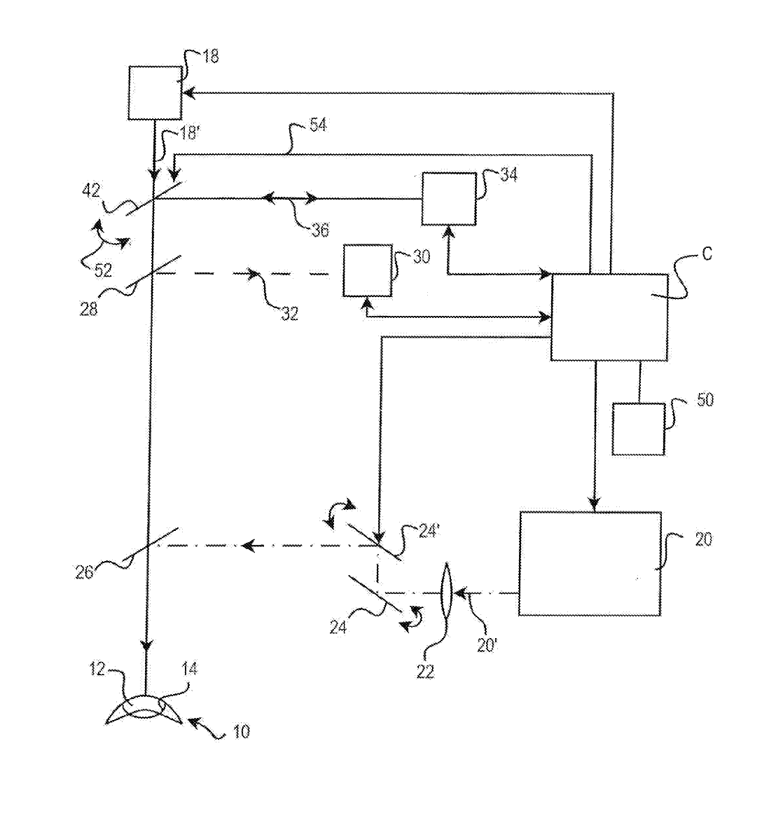

### CROSS-REFERENCE TO RELATED APPLICAITON

This application is a United States national phase application of co-pending international patent application No. PCT/EP2008/005334, filed Jun. 30, 2008, the disclosure of which is incorporated in its entirety herein by reference.

### BACKGROUND

The invention relates to an apparatus for ophthalmological, in particular refractive, laser surgery.

### SUMMARY

Surgery on the human eye encompasses numerous treatment methods in which laser radiation is directed onto the eye in order to obtain an indicated aim of treatment as a consequence of the interaction of the radiated laser radiation with the eye. In the case of refractive laser surgery, the aim of treatment is an alteration, by means of the laser radiation, of the imaging properties of the optical system constituted by the eye. Since the cornea, above all, is crucial for the imaging properties of the human eye, in many cases refractive laser surgery on the eye includes a treatment of the cornea. By targeted introduction of incisions and/or by targeted resection of material a change of shape of the cornea is brought about; one therefore also speaks of a reshaping.

A known example of a reshaping of the cornea for the purpose of altering its refractive properties is LASIK (laser in-situ keratomileusis). In the case of LASIK, a superficial cover disc, which in specialist circles is generally designated as a flap, is cut out of the cornea. On a part of its edge in a hinge region the flap is still connected to the corneal tissue situated alongside, so that it can be folded aside and later folded back again without difficulty. For the purpose of producing the flap, in prior practice two methods in particular have found application, on the one hand a mechanical method by means of a microkeratome, and on the other hand a laser-technology method, wherein by means of femtosecond laser radiation (i.e. pulsed laser radiation with a pulse duration within the fs range) a planar depth incision is introduced into the cornea, which, except for the hinge region, is guided out to the corneal surface. After the flap that has been produced has been folded away, a resection (ablation) of material from the stroma which has been exposed in this way is effected in accordance with a predetermined ablation profile. The ablation profile specifies at which point of the cornea how much tissue is to be resected. It is calculated in such a way that after the ablation the cornea has a shape that is optimal for the eye being treated, and the previously existing optical aberrations of the eye are corrected as extensively as possible. For the calculation of the ablation profile, suitable methods have been available to specialists in the field for quite a long time. An excimer laser, for example, with a radiation wavelength in the UV region, at approximately 193 nm, finds application for the ablation.

Once the ablation profile for the eye to be treated has been determined, a calculation is subsequently made regarding how the desired resection can best be achieved with the laser radiation (therapeutic radiation) available. The laser radiation that is used is normally pulsed radiation. It is therefore a question of calculating a sequence of laser pulses in space and time, which—in interaction with the cornea, in particular the stroma—brings about the desired reshaping of the cornea.

Beam-guidance means, in order to guide a laser beam over the eye to be treated in such a way that the desired spatial and temporal sequence of the laser pulses arises, are known as such in the state of the art. In particular, the beam-guidance means may include a deflecting unit, also known as a scanner, serving for deflection of the therapeutic laser beam in the transverse direction (x-y direction), as well as focusing optics for focusing the laser beam at a desired vertical position (z-position). The deflecting unit may, for example, include one or more galvanometrically controlled deflecting minors.

The aforementioned beam-guidance means are controlled by means of a program-controlled computer in accordance with the ablation profile. Since the invention is by no means restricted to use in the course of LASIK but may find application in numerous other laser-surgery interventions in respect of the eye, in the following a treatment profile will generally be referred to, in accordance with which the beam-guidance means are controlled. For incisional interventions, in which incisions are introduced into the cornea or into another component of the eye, the treatment profile may represent an incision profile that specifies at which point how deep an incision is to be made.

The human eye is not a stationary object but is constantly executing movements. There are varying types of eye movements, which, in part, proceed on varying timescales and with varying amplitudes. What is important is solely the observation that the eye is never at rest. This also applies when an attempt is being made to fix the gaze onto a certain predefined object; even then, unavoidable fixation movements arise.

For the purpose of registering the aforementioned eye movements, systems are known for eye-movement tracking or gaze-movement registration (eye-trackers). These systems normally include at least one camera which is directed onto the eye and which records sequences of images of the pupil, including the surrounding iris. By subsequent evaluation of the image sequences by means of suitable image-analysis algorithms, the current position of the pupil and the course of motion of the pupil can be established. In particular, the pupillary centre is drawn upon in this connection. By orientation of the treatment profile with respect to the pupillary centre which is monitored by camera technology in this way, or with respect to a point derived from said centre, the desired spatial sequence of laser pulses can be reliably routed onto the correct points of the eye region to be treated, despite the unavoidable eye movements.

The basis for the ascertainment of a suitable treatment profile is normally a survey of the eye in its actual state. For the refractive corneal treatment, for example, normally knowledge of at least the topography and thickness of the cornea is required. Knowledge of other or further parameters of the eye may be required for the treatment to be carried out—for instance, the depth of the anterior chamber, the thickness of the lens, the total depth of the eye, and such like. Parameters of such a type are measured not only prior to the start of the treatment but also, at least partly, during or/and after the treatment, for example in order to log the course of the treatment and, where appropriate, to intervene in controlling manner in the course of the treatment and in order to examine the result of treatment.

For non-contacting surveying of eye parameters such as, in particular, the corneal thickness, optical-coherence interferometric measuring devices have been available for some time, which operate, for example, in accordance with the principle of optical low-coherence reflectometry (OLCR) or optical coherence tomography (OCT). These measuring devices operate with low-coherence, broadband radiation and permit structures of the eye (or generally of the biological tissue to be surveyed) to be surveyed with a resolution in the region of 1 μm and finer. Optical-coherence tomography is an imaging process that enables the generation of incision images. Optical low-coherence reflectometry is suitable, on the other hand, in particular for punctual measurements of a thickness dimension or depth dimension of the eye, such as, for instance, the corneal thickness (pachymetry).

The measurement of the corneal thickness (or of another thickness dimension or depth dimension of the eye) is made difficult by the aforementioned eye movements. If repeated measurements of the corneal thickness are desired, for instance during an ongoing operation, then the measurement should be made, as far as possible, always at the same point of the cornea or at least within a certain region of the cornea (acceptance region) in which reliable results of measurement can be expected. Fixation movements of the eye, however, may have the result that this acceptance region disappears from the ‘field of view’ of the pachymetric measuring instrument and for this reason the measurement has to be interrupted. For the physician this has the consequence that no measured data can be registered. He/she must then either track the patient's head or not carry out any further measurement.

### DETAILED DESCRIPTION

The object of the invention is to simplify measurements of thickness or depth that become necessary within the scope of a laser-surgery treatment of the eye—be it before, during or after the operation—and to make them more reliable.

With a view to achieving this object, in accordance with the invention an apparatus for ophthalmological, in particular refractive, laser surgery is provided, comprising
a first radiation source for providing a treatment laser beam,
first beam-guidance means for location-controlled and time-controlled guiding of the treatment laser beam over an eye to be treated,
a camera for recording an image of the eye to be treated,
an evaluating and control arrangement evaluating the image data of the camera for the purpose of detecting eye movements and
an optical-coherence interferometric measuring device for measuring a thickness dimension or depth dimension, in particular a corneal thickness, of the eye, the measuring device including a second radiation source providing a measuring beam and also second beam-guidance means in order to direct the measuring beam onto the eye.

In accordance with the invention, the second beam-guidance means include at least one beam-guidance element which is movably arranged for the purpose of changing the beam position of the measuring beam, the evaluating and control unit being set up to control the beam-guidance element in a manner depending on registered eye movements in such a manner that the position of the measuring beam follows the eye movements.

The invention consequently teaches the concept of using the data of an eye-tracker registering the eye movements for the purpose of controlling the measuring device, so that in the course of implementation of the measurement the measuring beam always impinges substantially on the same point of the corneal surface or at least in the same region of the corneal surface. The tracking, automated in such a way, of the measuring beam in a manner depending on the detected eye movements enables a very large number of measurements in temporally short succession, allowing a precise documentation or/and control of the course of treatment. The laboriousness of the manual tracking in prior systems, on the other hand, has the result that, again and again, large time-intervals arise between consecutive measurements. In addition, the solution according to the invention guarantees a high reliability of measurement, since the coupling of the measuring device to the eye-tracker permits a constantly precise orientation of the measuring beam onto a predetermined point or onto a predetermined region of the eye.

The requirement to control the beam-guidance element of the measuring device in such a way that the position of the measuring beam follows the eye movements does not necessarily imply a continual, continuous tracking of the measuring beam to currently registered eye movements. As already mentioned, an acceptance region may have been predetermined, within which the measuring beam on the corneal surface may be located, without this having significant effects on the accuracy of measurement. However, as soon as the measuring beam leaves the acceptance region a tracking of the measuring beam takes place, so that the position thereof again lies within the acceptance region. In this connection, additional boundary conditions may have to be satisfied, which are intended to prevent unnecessary tracking movements of the measuring beam. For example, one such boundary condition may be that the measuring beam must have left the aforementioned acceptance region for at least a predetermined period of time before a tracking of the measuring beam takes place. Brief outliers can be filtered out in this way. It should at least be ensured that at the times at which the measuring device is carrying out a measurement the measuring beam is substantially oriented towards the predetermined point or the acceptance region. Since tracking of the measuring beam is possible comparatively quickly, only shortly before the intended measurement it is conceivable to adjust the position of the measuring beam in a manner depending on the then current position of the eye or of the pupil. It is, of course, similarly possible to perform operations for tracking the measuring beam also when no measurement is imminent.

In a preferred embodiment, the movably arranged beam-guidance element is a deflecting minor, from which the measuring beam reaches the eye without further deflection on a minor. Provided that the deflecting mirror lies in the path of a further beam of light of the laser-surgery apparatus that is directed onto the eye, the deflecting mirror is expediently a partially transmitting minor. For example, such a further beam of light may be a fixing light beam emitted from a source of fixation light.

For the purpose of changing the position of the measuring beam on the eye, the deflecting mirror may be arranged to be capable of tilting about at least one tilt axis. Alternatively or in addition, it may be arranged to be rectlinearly adjustable along at least one linear direction.

It has already been mentioned that it is not necessary in every case to adapt the position of the measuring beam continually to each registered eye movement. Accordingly, in accordance with a preferred further development of the invention the evaluating and control unit may have been set up to track the measuring beam to the eye movements by control of the beam-guidance element only when the registered eye movements satisfy at least one predetermined condition. Such a predetermined condition for the tracking of the measuring beam may be, for example, that the eye has moved by at least a predetermined extent in relation to a reference position. The reference position may, for example, relate to the position of the pupillary centre. Current eye-trackers on the market and their image-evaluation software are capable of calculating the current position of the pupillary centre from the registered image data. For example, the position of the pupillary centre at the start of the laser surgery may be used as a first reference position, and the measuring beam can be oriented relative to this first reference position. So long as the pupillary centre is subsequently located within a predetermined region (defined, for example, by a predetermined radius) around the first reference position, no tracking of the measuring beam takes place. If, on the other hand, the pupillary centre is remote from the reference position by more than the predetermined radius, a tracking of the measuring beam can take place. This may, for example, happen in such a way that a new reference position is established on the basis of the current information about the position of the pupillary centre, and the measuring beam is now oriented with respect to the new reference position. The new reference position may, for example, be an averaged position of the pupillary centre after leaving the previous acceptance region. Depending on the new reference position, a new acceptance region is then established, once again in the form, for example, of a circumscribed circle around the new reference position with a predetermined radius. It will be understood that other procedures and conditions for the tracking of the measuring beam in a manner depending on the movements of the eye are possible at any time.

The invention will be elucidated further in the following on the basis of the single drawing. The latter shows, in schematic block representation, an exemplary embodiment of an apparatus for refractive laser surgery on the eye. In the drawing, an eye to be treated by laser surgery, for example refractive laser surgery, is schematically indicated at 10. The cornea of the eye 10 and also the pupillary margin are shown at 12 and 14, respectively.

The laser-surgery apparatus that is represented exhibits, in a manner known per se, a source 18 of fixation light which emits a (weak) beam 18′ of fixation light and is sighted by the patient for the purpose of fixing the eye.

Furthermore, the laser-surgery apparatus includes a therapeutic laser 20 which emits treatment radiation 20′ which is directed onto a pair of scanner minors 24, 24′ via a lens 22 and is routed onto the eye 10 via a partially transmitting deflecting mirror 26. For a LASIK treatment, the laser 20 may be, for example, an excimer laser, the radiation wavelength of which lies in the UV region, for instance at 193 nm. It will be understood that for other aims of treatment other treatment wavelengths may also be used if desired, also in the infrared region. The scanner minors 24, 24′ are, for example, galvanometrically controllable and are controlled together with the laser 20 by a program-controlled computer C in accordance with a previously calculated treatment profile. The computer C represents an evaluating and control unit in the sense of the invention.

The laser-surgery apparatus possesses, furthermore, a device for tracking eye movements (eye-tracker). The eye-tracker includes a camera 30, with which via a partially transmitting deflecting minor 28 in the direction of an arrow 32 images of the eye—in concrete terms, of the pupil and of the iris—can be recorded. The image data of the camera 30 are then evaluated in the computer C by means of image-analysis software, in order to track movements of the eye which the patient, as a rule, cannot avoid, despite the attempted fixation of the gaze onto the fixation beam 18′. The detected eye movements are taken into account by the computer C in the control of the scanner minors 24, 24′, in order in this way to keep the treatment profile oriented as constantly as possible in relation to a predetermined reference point of the eye, which is situated, for example, on the corneal surface.

Integrated into the laser-surgery apparatus is, in addition, a measuring device 34 for optical low-coherence reflectometry (OLCR), which includes, in a manner known per se, a radiation source (e.g. SLED, ASE, supercontinuum laser), the measuring beam of which is directed onto the eye 10 via a partially transmitting deflecting mirror 42. The measuring device 34 receives radiation reflected from the eye 10 via the deflecting mirror 42 on the same path on which the measuring radiation of the measuring device 34 is emitted. This is illustrated by a double-headed arrow 36.

The measuring device 34 measures at least once, but preferentially several times, the corneal thickness and, if desired, one or more other thickness dimensions or depth dimensions (e.g. the depth of the anterior chamber) of the eye 10 to be treated. Expediently a measurement of the corneal thickness is effected at least once before the start of the laser surgery and one further time after conclusion of the laser surgery. Preferentially, measurements also take place continually during the laser surgery, for example at predetermined regular intervals. The measuring device 34 supplies its measurement data to the computer C which is able to represent the results of measurement numerically or/and graphically, for example on a display unit 50. Where required, the computer C may also have been set up to store the results of measurement and, subsequent to the operation, to bring about the printout of a measurement log which contains the results acquired within the scope of the consecutive measurements. However, the display of the results of measurement on the display unit 50 is advantageous, insofar as it permits the operator to monitor the progress of the treatment directly. Where required, the computer C or its control program may have been set up in such a way that it is receptive to corrective interventions of the operator and adapts the course of treatment appropriately. Such corrective interventions may be possible, for example, via an input apparatus which is not represented in any detail and with which the computer C is coupled.

The deflecting mirror 42, via which the measuring beam is coupled into the common beam path of the fixation light 18′ and of the therapeutic laser beam 20′, is movably arranged relative to the two other deflecting minors 26, 28, namely in the present exemplary case in swivelling manner, as indicated by a double-headed arrow 52. In this connection the deflecting minor 42 is capable of swivelling about at least one tilt axis which is substantially parallel to the x-y plane of the laser-surgery apparatus. According to the current conception, the x-y plane is the plane that is parallel to the direction of incidence of the therapeutic laser beam 20′ (z-direction). By tilting of the deflecting minor 42 about a tilt axis situated in such a manner, the part of the measuring beam impinging on the eye 10 can be swivelled, and hence the incident position of this laser beam can be changed. Expediently the deflecting minor 42 is capable of swivelling about two mutually perpendicular tilt axes, each situated substantially parallel to the x-y-plane, so that the position of the measuring beam on the eye 10 can be changed two-dimensionally. For the purpose of adjusting the deflecting mirror 42, galvanometric positioning means, for example, may be provided such as are known per se in the specialist field for the drive of scanner minors (for instance, the minors 24, 24′) . Other types of drive are, of course, not excluded, for instance electromotive or piezoelectric drives. The aforementioned positioning means of the deflecting minor 42 are controlled by the computer C, this being indicated in the drawing by a control connection 54.

As an alternative to a swivelling capacity of the deflecting minor 42, the latter may also be adjustable in the x-y plane without thereby changing its orientation relative to the x-y plane. In this way also, a displacement of the position of the measuring beam on the eye is capable of being brought about.

The computer C controls the deflecting mirror 42 in a manner depending on the position of the eye—more precisely, the position of the pupillary centre—ascertained from the image data of the camera 30. In this way the position of the measuring beam can be tracked in a manner depending on registered eye movements, guaranteeing that the thickness measurement always takes place in a region of the cornea that permits reliable statements about the corneal thickness. In order to give a numerical example, on the assumption of a spacing between the eye 10 and the deflecting minor 42 of, for instance, 445 mm, and on the assumption of a displacement of the corneal point sighted from the measuring beam by, for instance, 1.0 mm, the deflecting minor 42 should be tilted by, for instance, 0.065° in order to obtain the requisite inclination of the measuring beam by 0.13° which is necessary in order that the measuring beam continues to impinge substantially on the same corneal point.

1-6. (canceled)

7. Apparatus for ophthalmological, in particular treatment laser surgery, comprising:
a first radiation source for providing a therapeutic laser beam,
first beam-guidance system configured to control the location of the therapeutic laser beam over an eye to be treated,
a camera for recording an image of the eye to be treated,
an evaluating and control unit evaluating the image data of the camera for the purpose of recognising eye movements,
an optical-coherence interferometric measuring device configured to measure a depth dimension, in particular a corneal thickness, of the eye, the measuring device including a second radiation source providing a measuring beam and also second beam-guidance system controllable to direct the measuring beam onto the eye,
wherein the second beam-guidance system includes at least one movably arranged beam-guidance element for changing the beam position of the measuring beam and in that the evaluating and control unit has been set up to control the beam-guidance element in a manner depending on registered eye movements in such a manner that the position of the measuring beam follows the eye movements.

8. Apparatus according to claim 7, characterised in that the movably arranged beam-guidance element is, in particular, a partially transmitting deflecting mirror from which the measuring beam reaches the eye without further deflection on a mirror.

9. Apparatus according to claim 8, characterised in that the deflecting mirror is arranged to be capable of tilting about at least one tilt axis.

10. Apparatus according to claim 8, characterised in that the deflecting mirror is arranged to be linearly adjustable.

11. Apparatus according to claim 7, characterised in that the evaluating and control unit has been set up to track the measuring beam to the eye movements by control of the beam-guidance element only when the registered eye movements satisfy at least one predetermined condition.

12. Apparatus according to claim 11, characterised in that a predetermined condition for the tracking of the measuring beam is that the eye moves by at least a predetermined extent in relation to a reference position.

13. A method of performing ophthalmic surgery, comprising:
providing a first radiation source operable to generate a first therapeutic laser beam, a second radiation source operable to generate a measuring beam;
evaluating the position of the eye to be treated;
directing the measuring beam on to the eye at a first location through a measurement beam guidance arrangement based at least in part on the position of the eye;
measuring a thickness dimension of at least a portion of the eye at the first location; and
guiding the first therapeutic laser beam on to the eye at the first location through a therapeutic beam guidance arrangement that is separate from the measurement beam guidance arrangement.

14. The method of claim 13, wherein said evaluating and said directing steps operate together such that the position of the measuring beam follows the eye movements.

15. The method of claim 13, wherein said measuring is performed by an optical-coherence interferometeric measuring device.
